(19) United States
(12) Plant Patent Application Publication
(10) Pub. No.: US 2017/0118881 P1
(43) Pub. Date: Apr. 27, 2017

(54) Cherry tree named 'PA6UNIBO'

(50) ;

(71) Applicant: Alma Mater Studiorum - Università di Bologna, Bologna (IT)

(72) Inventors: Stefano Lugli, Bologna (IT); Ricardo Correale, Bologna (IT); Michelangelo Grande, Bologna (IT)

(73) Assignee: Alma Mater Studiorum - Università di Bologna, Bologna (IT)

(21) Appl. No.: 15/330,470

(22) Filed: Sep. 22, 2016

Related U.S. Application Data
(60) Provisional application No. 62/284,255, filed on Sep. 23, 2015.

(51) Int. Cl. A01H 5/08

(52) U.S. Cl. CPC A01H 5/085
USPC 800/298; PLT/181

(57) ABSTRACT

‘PA6UNIBO’ is a new cherry tree variety selected for its vigorous growth and excellent yield, as well as its attractive dark red, flavorful fruit. ‘PA6UNIBO’ cherry ripens 10 to 12 days after ‘Burlat’ cherry.

Variety denomination: ‘PA6UNIBO’.

Genus and species: Prunus avium L.

### BACKGROUND AND SUMMARY OF THE VARIETY

‘PA6UNIBO’ cherry tree, originally derived from a seedling of unknown parentage and called Selection DCA BO B5 D20, was first discovered in 2000 at Vignola, Modena Province, Italy and planted in 2002 at the same location. The first crop was obtained in 2004. ‘PA6UNIBO’ was first propagated on ‘Colt’ (U.S. Plant Pat. No. 4,059) and ‘CAB 6P’ (not patented) root stocks of differing vigor in trial fields of Bologna University to test growth and yield performance under differing training systems and planting densities, and was found to have the properties proper to a promising new cultivar for the market. ‘PA6UNIBO’ picks 10 to 12 days after ‘Burlat’ (not patented) (8 to 10 days before Bing).

Compared to Celeste® ‘Sumpaca’ (not patented) cherry tree, the fruit of ‘PA6UNIBO’ are larger, heart-shaped, of firmer flesh, with longer stem, more uniform ripening, and higher sugar content.

‘PA6UNIBO’ cherry tree was selected for its high qualities, including tree growth and yield performance, and fruit appearance and excellent taste-flavor properties.

### BRIEF DESCRIPTION OF THE PHOTOGRAPHS

FIG. 1 shows detail of full bloom of ‘PA6UNIBO’ cherry tree;

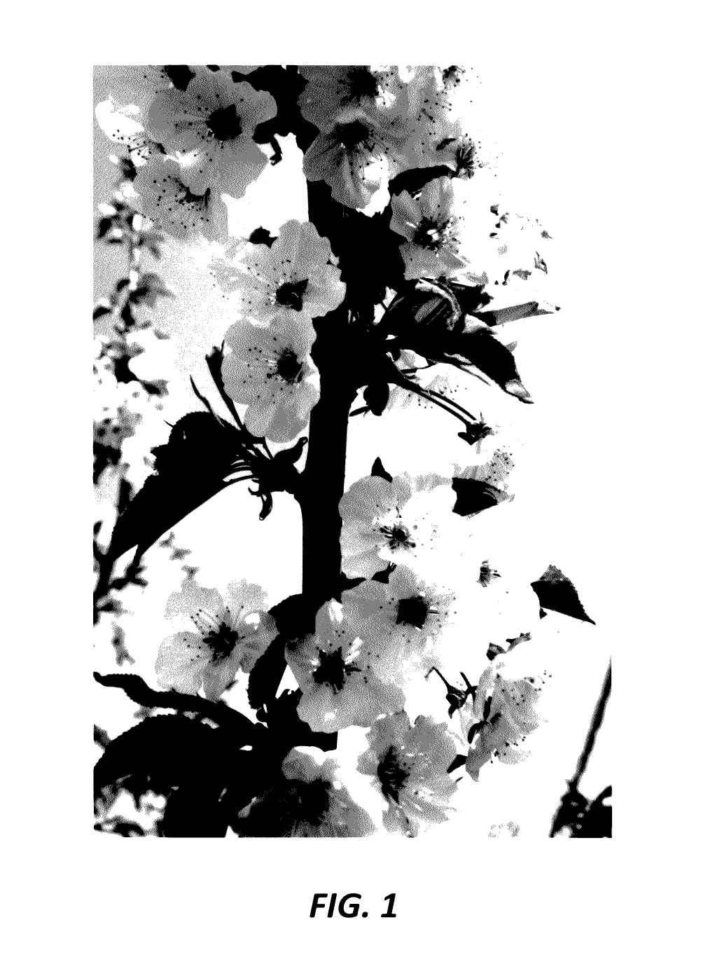

FIG. 2 shows the habit of the ‘PA6UNIBO’ tree; and

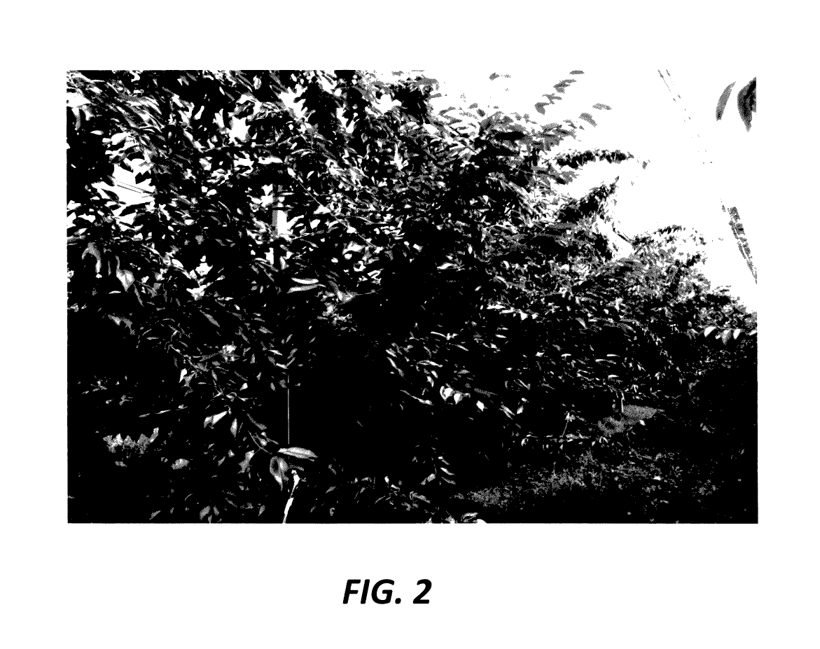

FIG. 3 shows detail of ‘PA6UNIBO’ fruit and leaves.

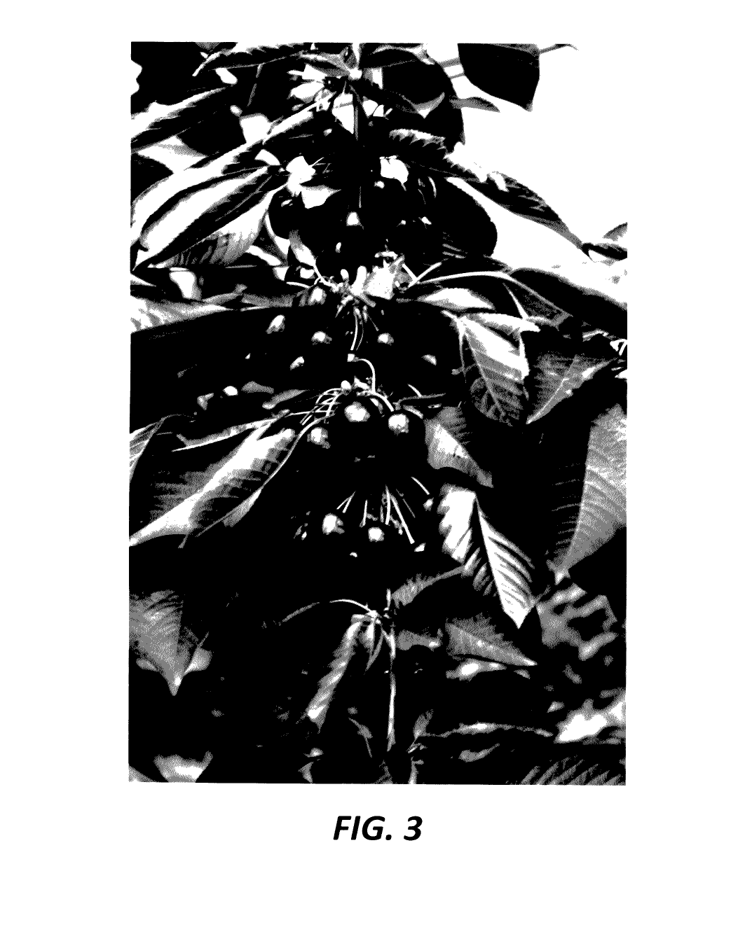

### DETAILED BOTANICAL DESCRIPTION OF THE VARIETY

The following is a detailed botanical description of ‘PA6UNIBO’ cherry tree, based on observations of 8-year-old trees made during the 2015 growing season at Vignola, Modena Province, Italy. All colors are described according to The Royal Horticultural Society Color Chart. It should be understood that the botanical and analytical characteristics described will vary somewhat depending upon cultural practices and climatic conditions, and can vary with location and season. Quantified measurements are expressed as an average of measurements taken from a number of individual plants of the new variety. The measurements of any individual plant, or any group of plants, of the new variety may vary from the stated average
Tree:
   Vigor.—High.
   Height.—4.5 meters.
   Spread.—3 meters.
   Growth habit.—Semi-upright.
   Canopy density.—Medium.
   Trunk diameter at about 25 cm height, measured at about 5 cm above graft joint.—
   Trunk color.—Reddish-brown 200C.
   Bark texture.—Slightly rough.
   Tendency toward alternate bearing.—No.
   Winter hardiness.—Unknown, no cold damage found in Po Valley district where grown.
   Chilling requirement.—Unknown.
Branch:
   Length.—250 cm to 300 cm.
   Diameter.—5 cm to 7 cm.
   Crotch angle.—35° to 50° depending on training system.
   Bark color.—Reddish-Brown 200C.
   Bark texture.—Slightly rough.
Current year shoot:
   Length.—50 cm to 60 cm.
   Color.—Reddish-brown 200D with fairly widespread gray 201A overtone.
Flower bud:
   Number per spur.—3 to 4.
   Shape.—Sub-globose.
   Length.—About 1 cm.
   Diameter.—Under 1 cm.
   Color.—Reddish perules 187C.
Flower:
   Number per cluster.—Minimum 8, maximum 10 per spur; 2 to 3 per bud. Bloom is abundant.
   Relative position of petals.—Petals overlap slightly.
   Diameter.—35 mm to 42 mm.
   Depth.—10 mm to 13 mm, measured as distance from receptacle to maximum petal aperture.
   Pollen.—Present, color yellow 12C.
Sepal:
   Length.—6 mm to 8 mm.
   Shape.—Slightly pointed.
   Width.—4 mm to 5 mm.
   Apex.—Pointed.
   Base.—Flat.
   Margin.—Regular. Color — upper surface: Pale green 138C Color — lower surface: Pale green 138C with reddish venation 182A.
Petal:
   Number per flower.—5.
   Shape.—Circular.
   Length.—18 mm to 20 mm.
   Width.—14 mm to 16 mm.
   Apex.—Slightly sunken.
   Base.—Pointed.
   Margin.—Slightly undulated. Color — upper surface: White 155B Color — lower surface: White 155B.
Bloom:
   Date of bud burst.—Mar. 19 to 20, 2014.
   Date of first bloom.—Mar. 26, 2014.
   Date of full bloom.—Mar. 30, 2014.
Pedicel:
   Length.—32 mm to 36 mm.
   Diameter.—About 1.0 mm.
   Color.—Pale green 146D.
Pistil:
   Quantity.—1.
   Size.—15 mm to 18 mm.
   Color.—Pale green 145B.
Anthers:
   Quantity.—26 to 32.
   Length.—5 mm to 8 mm, outer anthers longer than inner anthers.
   Diameter.—About 1 mm.
   Color.—Orange-yellow 15B with white filament (155D).
Stigma:
   Quantity.—1.
   Size.—0.9 mm to 1.1 mm diameter.
   Color.—Pale green 145B.
Style:
   Quantity.—1.
   Size.—9 mm to 12 mm length.
   Color.—Green 150C at basal part, darker green 149C at distal part.
Ovary:
   Quantity.—1.
   Size.—4 mm to 6 mm.
   Color.—Green 144A.
Leaves:
   Length.—13.0 cm to 14.0 cm.
   Width.—7.0 cm to 8.0 cm.
   Shape.—Lanceolate-elliptical.
   Apex shape.—Pointed.
   Base shape.—Pointed.
   Margin.—Serrate, fine toothed. Color — upper surface: Dark green 137B Color — lower surface: Green-yellow 147B.
Petiole:
   Length.—3.8 cm to 4.5 cm.
   Diameter.—About 1.5 mm.
   Color.—Lower, green-yellow 148B; upper reddish 187B.
Fruit:
   Shape.—Heart-shaped.
   Diameter.—Mostly 30 mm.
   Height.—23 mm to 27 mm.
   Weight.—12 g to 14 g on average.
   Number per cluster.—4 to 8 per spur.
   Skin color.—Shiny dark red 187B tending to blackish-dark red 187A at advanced maturity.
   Lenticels density.—Medium-high.
   Skin thickness.—Thin.
   Skin texture.—Medium.
   Tendency to crack.—Medium-low.
   Anthocyanin coloration near skin.—None.
   Tenacity of flesh to stone.—Medium-low.
   Flesh color.—Red 53A.
   Flesh texture.—Fine.
   Juiciness.—High.
   Juice analysis.—17.6° Brix and 9.9 g/l malic acid (2011-2014 data).
   Stone size.—Medium.
   Stone shape.—Elliptical.
   Stone color.—Pale brown 165D.
Fruit stem:
   Length.—40 mm to 50 mm.
   Diameter.—Just over 1 mm.
   Color.—Green 143A.
Harvest:
   Maturity and harvest date range.—10 to 12 days after ‘Burlat’ (8 to 10 days before Bing).

1. We claim a new and distinct variety of cherry tree, substantially as illustrated and described herein.
